(19) United States
(12) Patent Application Publication
(10) Pub. No.: US 2008/0228151 A1
(43) Pub. Date: Sep. 18, 2008

(54) Transdermal drug delivery with controlled heat for treating musculoskeletal pain and/or inflammation

(76) Inventors: Jie Zhang, Salt Lake City, UT (US); Kevin S. Warner, West Jordan, UT (US); Randal Nelson, West Jordan, UT (US); Alan Vawdrey, Riverton, UT (US); Nathan Strong, Sandy, UT (US); Suyi Niu, Salt Lake City, UT (US); Michael D. Wessman, Salt Lake City, UT (US); Matthew Iverson, Salt Lake City, UT (US)

Correspondence Address: THORPE NORTH & WESTERN, LLP., P.O. Box 1219, SANDY, UT, 84091-1219, US

(21) Appl. No.: 12/074,259

(22) Filed: Feb. 28, 2008

Related U.S. Application Data
(60) Provisional application No. 60/904,208, filed on Feb. 28, 2007.

(51) Int. Cl. A61F 7/08; A61K 9/00; A61K 31/192; A61K 31/196; A61K 31/167

(52) U.S. Cl. 604/290; 424/449; 514/570; 514/567; 514/626; 604/291

(57) ABSTRACT

Systems and methods for transdermal drug delivery with controlled heat are provided. Such systems can comprise a heating apparatus that includes an exothermic chemical composition, typically in the form of individual heating elements. The heating apparatus can be exposed to ambient oxygen through a cover. The cover can reduce the amount of ambient oxygen capable of contacting the chemical composition compared to when the cover is not present. In some embodiments, the heating apparatus can include more than one heating element. The systems can further include a drug-containing layer that includes a drug. The drug-containing layer can have a surface area of about 50 cm² to about 400 cm². The systems of the present invention can be deliver ketoprofen in an amount sufficient to produce a mean blood plasma concentration of ketoprofen in a human subject of at least 45 ng/ml within four hours after initial application of the system to a skin surface.

This application claims the benefit of U.S. Provisional Patent Application No. 60/904,208, filed Feb. 28, 2007, which is incorporated herein by reference.

### BACKGROUND OF THE INVENTION

Although oral NSAIDs (nonsteroidal anti-inflammatory drugs) have been used effectively to treat musculoskeletal pain and inflammation for decades, they have significant potential to cause bleeding in the gastrointestinal (GI) tract. Such bleeding has been linked with many deaths each year.

Attempts have been made to deliver anti-inflammatory and analgesic drugs directly into joints and muscles transdermally to treat musculoskeletal pain and inflammation of various causes, such as arthritis induced pain. For example, creams containing NSAIDs (nonsteroidal anti-inflammatory drugs) are marketed in Europe and Japan for treating joint pain. In this approach, a portion of the drug that has permeated across the skin is believed to enter the targeted tissues directly, without first entering the systemic circulation and then redistributing into the target tissues. Such “regional delivery” is believed to be able to deliver an effective amount of the drug into the target tissues while producing much lower drug concentrations in systemic circulation. Lower drug concentration in systemic circulation is believed to have lower potential of causing GI tract bleeding.

In accordance with this, it would be very desirable to provide systems and methods for administering NSAIDs in a manner that is less harmful to the patient, and specifically reduces the destructive effects such drugs have on a patient's GI tract. Further, it would be desirable to administer such NSAIDs in a manner that provides improved dermal drug delivery, as well as provide other additional benefits.

### BRIEF DESCRIPTION OF THE DRAWINGS

FIG. 1 is a schematic representation of a system for dermal delivery of a drug, in accordance with one embodiment of the present invention.

FIGS. 2A and 2B are a schematic representation of an alternative system for dermal delivery of a drug, in accordance with another embodiment of the present invention.

FIG. 3 is a single exemplary top view of a system shown schematically in FIGS. 1, 2A, and 2B; and

FIG. 4 is a graph of the results of an experiment wherein identical compositions were administered transdermally, both with and without a heating device.

### DETAILED DESCRIPTION OF THE PREFERRED EMBODIMENT(S)

Before the present invention is disclosed and described, it is to be understood that this invention is not limited to the particular process steps and materials disclosed herein because such process steps and materials may vary somewhat. It is also to be understood that the terminology used herein is used for the purpose of describing particular embodiments only. The terms are not intended to be limiting because the scope of the present invention is intended to be limited only by the appended claims and equivalents thereof.

As used in this specification and the appended claims, the singular forms “a,” “an,” and “the” include plural referents unless the content clearly dictates otherwise.

The terms “controlled heating” and “controlled heat” are defined as heat application that is capable of heating a skin surface to pre-determined narrow temperature range for a predetermined duration. A controlled heating device that can be used in accordance with systems and methods of the present invention can be configured to generate heat promptly when activated. Controlled heating can be achieved through special design of the heating apparatus. For example, controlled heating can be achieved through the use of a properly configured heating element(s) including an exothermic chemical composition. Considerations in generating controlled heat with an exothermic heating component assembly include proper ratios and chemical components used, as well as physical constraints put on the chemical components, e.g., limiting air flow or oxygen contact, spatial configuration of individual heating elements, conductivity of materials used with chemical components, etc. In one embodiment, the heating device can provide heat at a temperature greater than body temperature, but less than a temperature that would cause irreversible skin damage, e.g., burn the skin. An exemplary temperature range that can be implemented for use is from about 37° C. to about 47° C. In one embodiment, a more preferred temperature range can be from about 38° C. to 42° C.

As used herein, the term “active” when referring to a body surface, such as skin, indicates that the body surface regularly undergoes flexing, bending, and/or stretching. Such is the case with nearly all joints. For example, knees, elbows, fingers, necks, etc. Additionally, back muscles are considered active body surfaces because of the large amount of flexing, bending, and/or stretching. Areas of the skin that are not regularly stretched during normal activity are not considered to be “active.” For example, the scalp, arms and legs (other than at or near joints), etc., are not considered active body surfaces.

As used herein, the term “foil” refers to a primarily metallic material formed into a thin self-supporting sheet. The foil can comprise any metallic material; however, in one specific embodiment, the material can consist essentially of a metallic material, such as aluminum. Metal alloys are also included within this definition. The term “thin” when referring to a metal foil may be interpreted to mean any metal foil with a thickness from about 0.0001″ (0.1 mil, or 2.54 micrometer) to about 0.01″ (10 mil, or 254 micrometer).

This being stated, the present invention is drawn to systems for dermal delivery of a drug. Specifically, the present invention is related to a controlled heat assisted-transdermal drug delivery system for treating musculoskeletal pain or inflammation. The system comprises at least two components: a component that is capable of generating controlled heat (referred to as a “heating apparatus”), and a component that contains a drug formulation for transdermal delivery (referred to as a “drug component”). The heating apparatus can include at least one, and in many cases, at least two heating elements. The heating elements can each include an exothermic chemical composition for use in heat generation. Also, the heating elements can be selectively exposed to ambient oxygen through a cover. The cover can regulate the amount of ambient oxygen capable of contacting the chemical composition to a pre-determined (usually through simple experimentation based on the principles described herein) level, thereby providing desired and controlled oxygen exposure to the chemical composition. The combination of the chemical composition and the amount of oxygen delivered thereto can be matched to provide controlled heat, e.g., narrow temperature range, appropriate duration, etc. The systems can further include a drug component which comprises a drug-containing layer. Such layer can have a drug delivery surface appropriate for a predetermined use, and in some cases, can range from about 50 cm² to about 400 cm². In one aspect, the heating elements can collectively cover a total surface area from about 20% to about 80% of the surface area of the drug-containing layer. In other words, the area where the heating elements are at an interface with the drug-containing layer (either through a barrier or in direct contact), can be from about 20% to about 80% of the total lateral area (based on one side) of the drug-containing layer. The heating component and the drug component can be in one integrated unit, or can be made and stored separately and combined prior to, or upon use. The system can further comprise a means for affixing itself on the skin. Such means include, but are not limited to, a layer or sporadic use of adhesives and a strapping device. The present invention is related to a method of delivering drugs that combines the effects of transdermal delivery with a heating system. Such combination can harness the benefits of both regional drug delivery and heating for treating musculoskeletal pain or inflammation. In order to develop combined drug delivery-heating systems that are efficacious, safe, and easy to use, many properties of the combined system should be carefully designed, and the present invention is related to such designs.

In one specific embodiment, a system for dermal delivery of a drug can comprise a heating apparatus and a drug-containing layer. The heating apparatus can comprise at least two heating elements, each including an exothermic chemical composition, wherein the heating element is exposed to ambient oxygen through a cover. The cover can be configured to control the amount of ambient oxygen capable of contacting the chemical composition. Regarding the drug-containing layer, this layer can include a drug and having a delivery surface with an area of about 50 cm² to about 400 cm² and a drug.

In another embodiment, a system for dermal delivery of a drug can comprise heating apparatus and a drug-containing layer. The drug-containing layer can comprise an NSAID and can have a drug delivery surface having an area of about 50 cm² to about 400 cm². The drug-containing layer can be formulated to provide dermal delivery of the NSAID. The heating apparatus can include at least one heating element with an exothermic chemical composition. The heating element can be configured to be exposed to ambient oxygen through a cover which controls the amount of ambient oxygen capable of contacting the chemical composition to provided controlled heating. The heating apparatus can also be configured to be connected to or disposed proximate to the drug-containing layer such that when placed on a human body surface of a subject experiencing musculoskeletal pain or inflammation, the controlled heating from the heating apparatus and the dermal delivery of the NSAID from the drug-containing layer provides greater relief to the subject defined by less inflammation or less pain retained by the subject compared to when either dermal delivery of the NSAID or application of the controlled heating is administered alone. In one embodiment, the heat and drug in combination can generate a synergistic effect.

In another embodiment, a system for treating musculoskeletal pain or inflammation can comprise an elastic air impermeable cover including a plurality of holes therethough and at least two heating elements positioned beneath the cover. The system can also include a polymeric layer positioned beneath the heating elements, and sealed to the cover to provide one or more chamber for the heating elements. A metal barrier can be positioned beneath the polymeric layer, and a drug-containing layer including a drug delivery surface having an area of about 100 cm² to about 250 cm² can be positioned such that it is separated from the heating elements by the metal barrier. The metal barrier can be configured to prevent migration of the drug into the heating apparatus. Additional layers can also be present, such as a second polymeric layer interposed between the metal barrier and the drug-containing layer.

In another embodiment, a system for treating musculoskeletal pain or inflammation can comprise a heating apparatus comprising at least 5 heating elements, each with a surface area on one side from about 12 cm² to 14 cm². The heating elements each include an exothermic chemical composition, wherein each heating element is exposed to ambient oxygen through a cover. The cover can comprise a material that is air-impermeable, but which has at least six holes associated specifically with each of the at least five heating elements, wherein the holes have a diameter from about 0.065 inch to about 0.085 inch. In one embodiment, there are exactly five heating elements, and each heating element has exactly six holes as described. A drug-containing layer comprising ketoprofen and having a surface area of about 150 cm² to about 200 cm² can be present. The heating apparatus can be configured to maintain the skin at a temperature from about 38° C. to about 42° C. for at least 6 hours.

In another embodiment, a method of treating musculoskeletal pain or inflammation can comprise applying a system to a body surface under which musculoskeletal pain or inflammation exists. The system can comprise a heating apparatus including at least two heating elements where each heating element includes an exothermic chemical composition. Each heating element can be exposed to ambient oxygen through a cover, which controls the amount of ambient oxygen capable of contacting the chemical composition. A drug-containing layer can include a drug and having a delivery surface with an area of about 50 cm² to about 400 cm² and a drug.

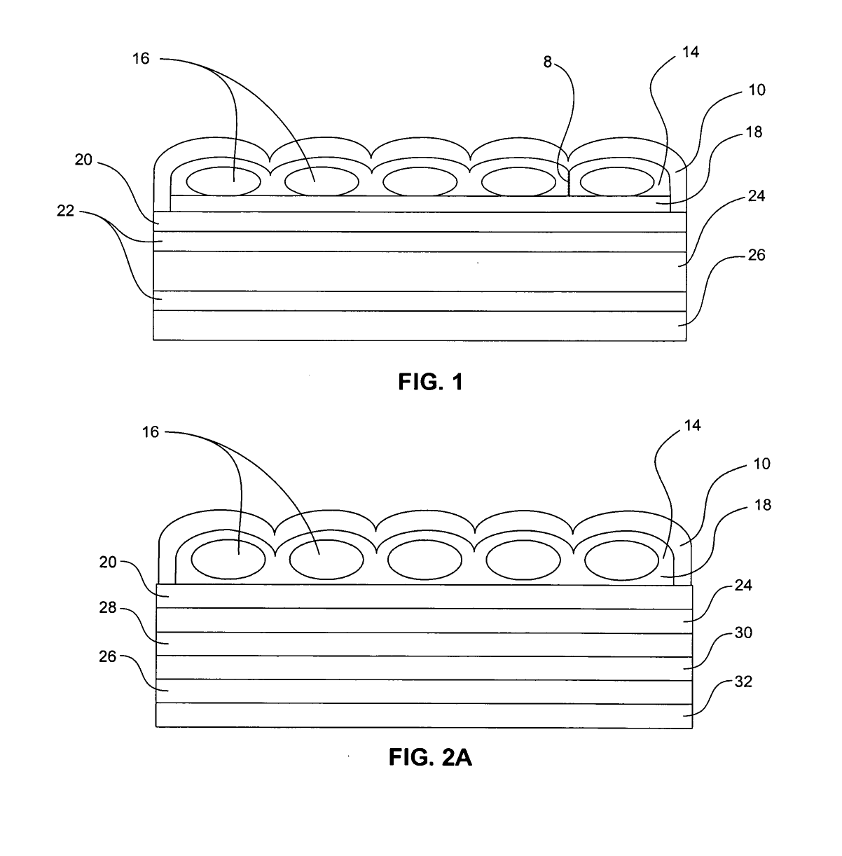

By way of example, FIG. 1 is a profile of one embodiment that illustrates one configuration of a device that can be used in accordance with embodiments of the present invention. The layers incorporated into one embodiment of the present invention include a stretchable polymeric air-impermeable foam or elastic material layer 10 with holes (not shown in this view) for allowing air to pass therethrough, heating elements comprising an air permeable enclosure 14 containing exothermic heating composition 16 (in the form of individual heating elements), a polymeric layer 18 that can be used to prevent transfer of water and salt, a layer of transfer adhesive 20, films of poly(ethylene acrylic acid) 22, a thin metal layer 24, such as a foil, and the drug-containing adhesive layer 26. A release liner (not shown in this embodiment) can be present to protect the drug-containing adhesive layer, as is known in the art. It is noted that an optional feature is shown at 8, where individual heating composition elements can be isolated from one another between layers 10 and 18. Each heating element composition element can be separated from one another, or can be together within a common chamber. Alternative configurations are also useable.

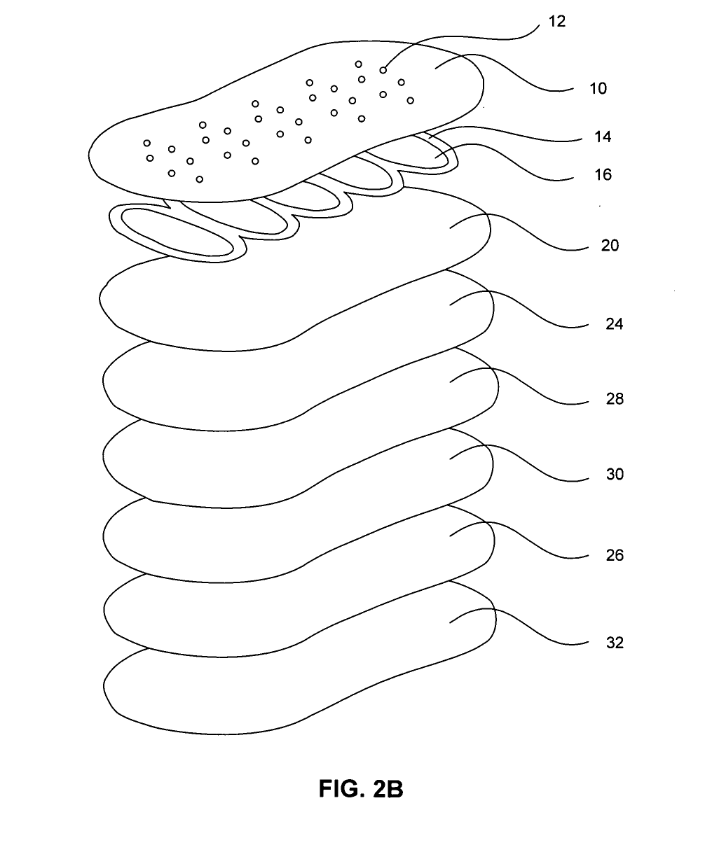

Alternatively, FIGS. 2A and 2B set forth an alternative embodiment, where FIG. 2A is a schematic side view and FIG. 2B is an exploded view. This embodiment is slightly different than the embodiment shown in FIG. 1. The layers incorporated into this embodiment include a stretchable polymeric air-impermeable foam or elastic material layer 10 with holes 12 for allowing air to pass therethrough. Heating elements are present and can comprise an air permeable enclosure 14 containing exothermic heating composition 16. A thin metal layer 24, such as a foil, is positioned immediately adjacent to a transfer adhesive 20 (such as an acrylic transfer layer), which can join the thin metal layer to the heating elements. One or two of polymeric layers 28, 30, can also optionally be present, such as ethyl acrylic acid and polyethylene, respectively, which are positioned between the thin metal layer and a drug-containing adhesive layer 26. A release liner 32, is also shown in this embodiment. Other optional layers can be present, or alternatively, some layers can be removed or repositioned, as would be known to one skilled in the art after considering the present disclosure. It is noted that though various thickness are shown in various embodiments, it is emphasized that these schematic drawings are not to scale, and various thickness can be used for each layer.

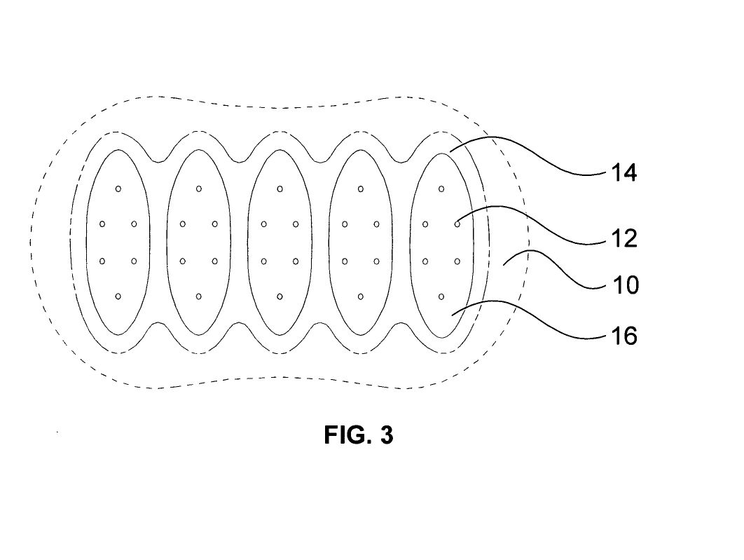

FIG. 3 shows an exemplary top view of the device of FIG. 1 or 2A. In this embodiment, the stretchable polymeric air-impermeable foam or elastic material layer 10 with holes 12 is shown. Additionally, the heating elements, including the air permeable enclosure 14 and the exothermic heating composition 16 are shown as outward facing depression in the elastic material.

One benefit of the system of the present invention is enhanced transdermal drug delivery by the controlled heating, as skin permeability to drugs can increase with increasing skin temperature. In addition, the controlled heating itself is also expected to reduce the musculoskeletal pain or inflammation. The combination of the transdermal delivery of a drug and the heat can boost the efficacy of either the drug or the heat alone. Further, in some embodiments, the selection and use of the drug and the amount and duration of the heat can provide synergistic effects.

A controlled heating device for use in accordance with embodiments of the present invention can generate and provide heat by one of a number of mechanisms. One mechanism involves generating heat by oxidation of certain metals, such as iron, through the use of an exothermic chemical composition. Such a mechanism can be configured to generate heat by an oxidation reaction between a component, e.g., iron, within the controlled heating device and oxygen in ambient air. U.S. Pat. No. 6,756,053, which is incorporated herein by reference in its entirety, describes such heating devices. Other heating mechanisms can also be used, such as heating by phase transition (such as phase transition of sodium acetate solutions) and electricity.

Although controlled heat in the heating apparatus can be generated by various mechanisms, in one aspect, formulations can utilize an exothermic oxidation reaction of metal. The heating apparatus, therefore, can include metal powder. Non-limiting examples of metal particulates, e.g., powders or filings, that can be used in the heating apparatus include iron and aluminum. As discussed, the heating apparatus can also have multiple heating elements, each containing an exothermic composition. An exothermic chemical composition can further include activated carbon, salt (such as sodium chloride), and water. In one aspect, a water-retaining substance, such as vermiculite, can be included in the composition. One issue with the exothermic chemical composition is that during the long storage time, gas (believed to be methane and hydrogen) is generated which puffs up the air tight container of the heating component (or the container containing the integrated heating and drug components), which can, in some cases, pose problems in storage and transportation. Certain amounts of sulfur-containing compounds, or salt thereof, such as elemental sulfur, sulfates, sulfites, sulfides, or thiosulfates, can reduce or eliminate this gas generation problem when included in the packaging.

Water content in the exothermic chemical composition can have an impact on the heating temperature profile of heating component. The weight ratio of water to the rest of the ingredients can be in the range of about 1:2 to about 1:8, and in some embodiments, from about 1.0:2.3 to about 1.0:4.5. It has been discovered in accordance with embodiments of the present invention that if the weight ratio of water to the rest of the ingredients is outside these ranges, the heating profiles (temperature, duration) are less desirable, though ranges outside of these are included within the scope of the present invention to the extent that they are functional. For example, these ranges can provide optimal heating over a more sustained period of time, e.g., above 38° C. for sustained durations).

The heating duration is dependent on the quantity and composition of the heat generating composition in each heating element. In order to obtain appreciable benefits from the controlled heating, the controlled heating can be configured to last sufficient duration. In some embodiments, the heating duration can be at least 2 hours, at least 6 hours, or even at least 10 or 12 hours. The drug delivery component of the systems of the present invention, e.g. transdermal patch, can be formulated to be left on the skin surface for a period of 8 to 14 hours. In one embodiment, the patch can be formulated to be left on the skin for a period of 10-12 hours. In yet a further embodiment, the patch can be formulated to be left on the skin for a period of 12 hours.

In one embodiment, the heating component comprises at least two, 2 to 20, or usually 3 to 8 heating elements. Each heating element can comprise a pre-formed bag formed of a material(s) that is substantially freely permeable to air and water. The heat generating composition resides inside the bag. In some aspects, each heating element can be formulated as part of a chambered heating element having a cover and having a certain number of holes associated therewith, e.g. located directly above as shown in FIG. 2 or 3. In one embodiment, each heating element can have from about 2 to 10 holes associated with it. Heating elements can be arranged in any manner that is conducive to providing heat to the system. In one aspect, the arrangement can be unstructured. In another embodiment, the heating elements can be formed into one or more rows. In more specific embodiments, the heating elements can be arranged into one, two, or three or more rows. In still another embodiment, the heating elements are arranged in pattern that is non-linear. For example, it may be desirable to arrange the heating elements in a manner that is ergonomically configured for application over a specific joint. For example, without limitation, a knee or elbow joint may benefit from radially positioned heating elements that surround the knee cap or elbow.

Each heating element can be enclosed to form individual chambers, or all can be collectively configured in a single chamber. Such chambers can have at least one side defined by a material that is permeable to air, either by use of a material that is inherently permeable or by placing holes in an otherwise impermeable material. Further, any number of heating elements can be included in a chamber. For example, a system with 10 heating elements can have 1 chamber with all 10 elements, 2 chambers with 5 elements each, 5 chambers with 2 elements each, or chambers with 1 element each. Additionally, the chambers need not be evenly defined. Continuing with the example of a system with 10 heating elements, then, there can be 3 chambers holding 2, 5, and 3 heating elements, or any other chamber-heating element arrangement.

The plurality of heating elements in the heating apparatus provides at least two advantages: minimizing sagging of the heat generating composition which tends to be worse in larger unrestricted containers, and providing better fit and flexibility if used on joints or other skin areas subject to bending. However, too many heating elements in the heating component would increase the cost and make the manufacturing process more expensive. Therefore, in one embodiment, the number of heating elements in the heating component can be from about 2 to about 20, and further from about 3 to about 8, or even 3 to 6. In further embodiments, 5 heating components, or alternatively 6 heating components can be used.

The size of each heating element can also be important. As each of the heating elements is not flexible itself, too large a size of each heating element can make the product uncomfortable to wear, particularly with active tissue. On the other hand, it can be difficult to fine-tune the number and size of holes in the cover for very small heating elements. They may also be more expensive to make. Therefore, the surface area of each heating element in the systems of the current invention, according to one aspect, can be from about 5 cm² to about 25 cm², or in some embodiments, from about 8 cm² to about 18 cm², or even from about 11 to about 15 cm².

In some embodiments, the heat-generating composition in each heating element has access to ambient oxygen only through the holes in a cover that is made of air-impermeable material. In this way, the flow rate of oxygen from ambient air into the heat generating composition, which is one of the factors that determine the heating temperature, is controlled by the size and number of holes on the cover. A unique feature of the heating component in some of the embodiments of the current invention is that there are pre-designed numbers of holes with pre-designed diameter in the cover over each heating element (both are usually selected to provide desired heating profile through experimentation). Further, the holes can be specifically associated with a particular heating element, thus fine tuning the oxygen flow.

With this design, the oxygen flow into each heating element can be designed, fine-tuned, and controlled so that the heating temperature can be more precisely and consistently in the desired range. In contrast, many of oxidation-based heating products on the market, such as hand warmers and ThermaCare, use a layer of air-permeable fabric material to cover the heat generating composition. In that design, it would be difficult to fine-tune the oxygen flow rate, and thus the heating temperature, because the oxygen flow rate is dependent on the physical characteristics of the particular material of use. In addition, the air permeability of the fabric material from batch to batch can be inconsistent and difficult to control, which can lead to variable and hard-to-control heating temperature variations. That said, it may be beneficial in some embodiments to utilize such permeable materials for the cover of heating elements. In one embodiment, the cover of the heating apparatus can be a sheet of material having a pre-determined air permeability that slows down oxygen flow from ambient air to each heating element.

Although the holes on the cover can be made any size, current manufacturing equipment and practices can limit the practical size of the holes. As such, holes smaller than 0.05 inch on a plastic sheet whose one side is coated with an adhesive, are not particularly cost-effective and can introduce unwanted variation and defects in the material because the small circular punched-out pieces tend to stick on the sheet and can block the holes. Therefore, according to one design in the current invention, the holes have at least 0.05 inch diameters in the air-impermeable cover for each heating element.

The percentage of surface area of the air-impermeable cover of each heating element that is made up of holes has to be in a relatively narrow range in order for the heating temperature to be in the range that is both therapeutically effective and harmless to the skin. This range can be from about 0.5% to about 2.5%, and in some embodiments, from about 1.0% to about 2.0%, and often, from about 1.2 to about 1.8%. It should be noted that the total surface area of the air-impermeable cover used in the above range calculations is defined as that directly accessible by the exothermic chemical composition. The surface area of the cover that is not directly accessible by the exothermic composition, such as the peripheral area beyond the surface area of the composition that is sealed onto the bottom sheet, is not included in the above range calculations.

Since the total surface area in the air-impermeable cover occupied by holes has to be in the aforementioned narrow ranges, larger holes will mean fewer holes. However, too few holes may mean inhomogeneous oxygen flow into the exothermic chemical composition, which is undesirable. Therefore, the holes cannot be too large. On the other hand, the holes cannot be too small either for the aforementioned manufacturing reason. The optimal range of the diameter of the hole can be from about 0.05 to about 0.12 inch, and in some embodiments, from about 0.065 to about 0.85 inch.

The drug component comprises a formulation that is designed to transdermally deliver the drug. The drug component may also comprise means of affixing itself (or the entire heating-drug combined system in the case of integrated systems) to the skin, such as a layer of adhesive. The formulation can be in the form of a patch, gel, paste, film, powder, oil, emulsion, adhesive, etc. While all of these dosage forms may be used in the current invention, the preferred dosage form is the drug-in-adhesive patch. The drug component may contain one, or a combination, of a variety of therapeutically effective drugs and appropriate enhancers. In a preferred embodiment, the drug of choice is an anti-inflammatory drug such as an NSAID, e.g. ketoprofen, diclofenac, salicylates, arylalkanoic acids, profens, fenamic acids, pyrazolidine derivatives, oxicams, COX-2 inhibitors, sulphonanilides, licofelone, omega-3 fatty acids, and combinations thereof. In one specific embodiment, the drug can comprise or consist essentially of ketoprofen. In another specific embodiment, the drug can comprise or consist essentially of diclofenac. However, the scope of this invention is not meant to be limited to this one drug class. For example, other drugs, such as local anesthetics, e.g. lidocaine, could also be beneficially delivered by the systems of the current application.

Although the drug delivered through transdermal absorption will eventually end up in the systemic circulation, a portion of the drug permeated across the skin is expected to enter the target tissues without passing through the systemic circulation. This mechanism allows a sufficient amount of the drug to enter the target tissues while producing systemic drug concentrations that are much lower than those produced by typical effective oral products containing the same drug.

In one embodiment, the target tissues are tissues in or around the knee. Drug molecules delivered across the skin adjacent to the knee, especially the area just above and just below the patella, have good chances to enter the target tissues directly. Drug molecules delivered across the skin sites too far from the knee have lower chances to reach the target tissues but will contribute to the systemic drug concentration (which one wants to minimize) just as much, or more. Based on these considerations, the surface area of the drug formulation exposable to skin in some of the embodiments of the current invention is designed to be from about 50 to about 400 cm², and in some embodiments, from about 75 to about 250 cm², and often, from about 150 to about 200 cm² to fully utilize the skin surface that favors direct drug entry into the target tissues without producing unnecessarily high systemic drug concentrations. For the same and other reasons, the shape of the drug formulation surface exposable to the skin is also optimized. The shape should be roughly a rectangle with rounded corners, with the length to width ratio in the range of 1.2:1 to 5:1. The rounded corners minimize edge lift during application, and can have the radius in the range of 2 to 10 cm. Too small a radius for the rounded corners would not fully minimize the edge lift potential, whereas too large a radius would compromise the delivery area and increase manufacturing waste. Such roughly rectangular shapes with rounded corners form an elliptical shape. Furthermore, the width around the center of the roughly rectangular, or elliptical, shaped drug formation surface can be narrower to accommodate the patella or other physical features of the targeted tissue area. Such variations to the shape of the drug formation surface can be used to modify the invention for use with specific joints or tissues.

Although the controlled heating can reduce pain and inflammation as well as increase drug penetration across the skin, covering all or close to all of the drug-skin contact area with heating element(s) can be undesirable because it allows no space between heating elements in the heating component and/or may cause an unacceptable level of moisture accumulation from sweating between the drug formulation and the skin. Such moisture accumulation can lead to discomfort and possible poor contact between the drug formulation and skin. Further, no or too little space between the heating elements can make the heating component rigid and uncomfortable to wear. On the other hand, heating too low a percentage of the drug formulation-skin contact area does not fully utilize the benefits of heating. To achieve the balance of the factors, in some of the embodiments, the total drug-skin contact surface area collective under all heating elements can be from about 20% to about 80% of the total surface area of the drug formulation exposable to skin. In another embodiment, this percentage is in the range of about 30% to about 70%.

The heating component and the drug component can be in one integrated system or in separate units but combined prior to or during use. However, an integrated system can need special designs for addressing issues unique to integrated systems. One of a potential need in an integrated system is prevention of drug migration into the heating component.

Although many air-impermeable membranes or tapes can be used as the cover for the heating elements, it is desirable to use the ones with good elasticity and/or stretchability. A heating component having elastic and stretchable membranes as the cover material for the heating elements tends to be more comfortable to wear and maintain better skin contact. However, more elastic materials often are more absorbent, which can be a problem if the drug formulation is not isolated from the absorbent materials in an integrated system. Therefore, in the integrated configuration, means to prevent chemical migration between the heating and drug components are often necessary. A barrier between the drug and the heating components can serve this purpose. For example, an elastic but very absorbent tape may be used to make the cover of the heating component, and a metal foil, or a laminate comprising a metal foil, can be placed between the drug and heating components to prevent drug migration from the drug component to the absorbent tape. However, a metal foil is typically less stretchable. This is not necessarily a significant problem for traditional transdermal patches which are not usually very stretchable themselves and are not usually applied to skin areas subject to significant stretch and bending. However, this may pose a serious problem for transdermal drug delivery systems designed to be used on highly stretchable skin surfaces such as that over back, neck, knees and other joints. One of the embodiments of the current invention uses the following novel approach to address this dilemma: a sheet of barrier material can be selected that is not necessarily stretchable or flexible but is fragile enough that it can be easily broken when stretched without significant resistance (hence without causing significant discomfort to the user). It should be noted that this approach is an option, not a limitation, for the systems and methods of the current invention.

The composition of the barrier layer can be aluminum, steel, copper, tin, nickel, an alloy of these metals, or a polymeric material known to form good barriers such as Barex®. Furthermore, the barrier layer can be a metal joined with a polymeric material on one or both sides, thus forming a multi-layered barrier. This additional polymeric film, as illustrated in FIG. 1, can help prevent tearing or breaking of the metal (foil) prior to use. Moreover, the polymeric film can provide a much more cosmetically appealing look for the system. Once this drug delivery system is in place on the patient it will be subjected to large amounts of stretching and flexing. At this point, the barrier layer may rip or tear and still be perfectly acceptable because the time it takes for the migration of significant amount of the drug is much longer than the duration of use by the patient. In other words, the miniscule amount of drug that may be lost during patient use will not affect the overall transdermal flux of the drug. The main purpose of the metal barrier layer is to prevent drug migration from the time of manufacture through packaging, shipping, and storage until application by the patient. After the product is removed from the packaging, the barrier layer may be compromised without adverse affect to the performance of said product.

Because the barrier can be compromised during wear, it may be desirable to use an extremely thin metal foil since thinner metal foils tend to be more readily breakable. Furthermore, the actual tearing of the metal layer is beneficial because the other layers of this system are then allowed to stretch and flex more freely. Therefore, the entire product is more stretchy and flexible due to the thinness and breakability of the metal barrier layer. This approach is novel because it is counterintuitive to design a product that is supposed to tear and break during use, particularly where a barrier layer is configured to break.

It should be noted that although a metal foil as described above may be desirable to use in some of the embodiments of the current invention, it is not a necessary element in all embodiments.

Another problem addressed by the metal barrier layer is the irregular distribution of heat over the surface of the system. If the barrier layer were polymeric then the surface heating profile may be unevenly distributed to follow the profile of the heating source. In one case, exothermic heating elements may provide higher temperatures directly underneath them, while areas in between heating elements would be cooler. In an ideal situation the heat should be distributed as evenly as possible over the surface to maximize the therapeutic surface area while reducing the possibility of burns from hot spots. The excellent heat transfer properties of the metal barrier layer address this issue by distributing the heat and thereby increasing the uniformity of surface heating. Therefore, the metal barrier layer can provide lateral homogenization of heat.

While a metal foil barrier can function well to stop the drug migration out of the drug layer and/or provide homogenization of heat, it can be subject to rust if it is in direct contact with the exothermic chemical composition which typically contains water and salt—elements that promote metal rusting. For example, the inventors noticed that the aluminum foil in an integrated ketoprofen transdermal-delivery system of the current invention was severely rusted by the heat-generating composition comprising iron powder, NaCl, water, activated carbon, vermiculite and sodium thiosulfate. To prevent rust, then, another barrier between the metal foil and the heat generating composition can be used. In one embodiment, the additional barrier can have low permeability to water and/or salt. One solution is to use an integral sheet of such a barrier material to completely separate the drug layer and the heating layer. However, that may reduce the stretchability and/or flexibility of the drug delivery system if the integral sheet itself is not elastic, which can be negative for applications on joints and muscles. Another approach is to place such a material only between each heating element and the metal foil layer. Alternatively, a fragmented sheet placed between the heating elements and the metal foil may also be used so that the movement between the heating elements is not significantly hindered by said sheet. In these embodiments, the barrier between the foil and the exothermic chemical composition does not limit the distances between the heating elements and thus does not significantly reduce the overall stretchability and flexibility of the entire system. It should be noted that the approaches of the multiple pieces or fragmented sheet as described above is not a necessary element in the embodiments of the present invention.

The system can deliver ketoprofen in at a rate such that the peak blood plasma concentration of ketoprofen in the patient occurs at from 6-11 hours after initial administration of the patch to the patient's skin surface. In one embodiment, the peak blood plasma concentration of ketoprofen in the subject can occur at about 7-10 hours after initial administration of the patch to the skin surface of the patient.

### EXAMPLES

The following example illustrates the embodiments of the invention that are presently best known. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following example provides further detail in connection with what is presently deemed to be the most practical and preferred embodiments of the invention.

### Example 1

### Transdermal Patch with Heating Assembly

A ketoprofen matrix material is prepared by dissolving ketoprofen into DuraTak pressure sensitive adhesive at a ketoprofen to DuraTak. The weigh ratio of the composition can be based on desired drug flux for a specific application. Independently, a 0.00035″ thick aluminum foil is coated on both sides with 0.00075″ layers of poly(ethylene acrylic acid) to give the aluminum added strength, provide enhanced adhesion to other layers, and prevent tearing during manufacturing, shipping, and storage. The aluminum composite material is then coated with a transfer adhesive on one side. The other side of the aluminum composite film is coated with the ketoprofen/DuraTak matrix material.

Separately, an exothermic chemical composition, e.g., iron filings, salt, activated carbon, filler, etc., is metered into multiple (2 to 20) bags, e.g., a nonwoven, fabric material that is essentially freely breathable with the ambient air, to form individual heating elements. The individual heating elements are contacted with a layer of PVDC film (on a bottom surface of the heating elements) which is sealed to the laminated foil. The heating elements are activated with water and then the entire system is covered with a flexible foam top configured 1 to 5 holes specifically associated with each heating element, where the surface area of the hole(s) is configured to provide controlled heat when the device is removed from its packaging at use. Upon opening of the packaging, oxygen flow will be controlled by the number and/or size of holes in the foam covering. It is noted that when the chemical composition is dosed with water, it is quickly sealed in air tight packaging, which will halt the exothermic reaction that begins when the water and oxygen contact the chemical composition, e.g., when all of the oxygen in the packaging is used up the reaction stops. Upon opening the packaging, the exothermic reaction resumes and the drug patch will be ready for use, providing controlled heat to the matrix layer and to the underlying active body surface.

### Example 2

### Improved Benefits of Heat with Dermal Drug Delivery

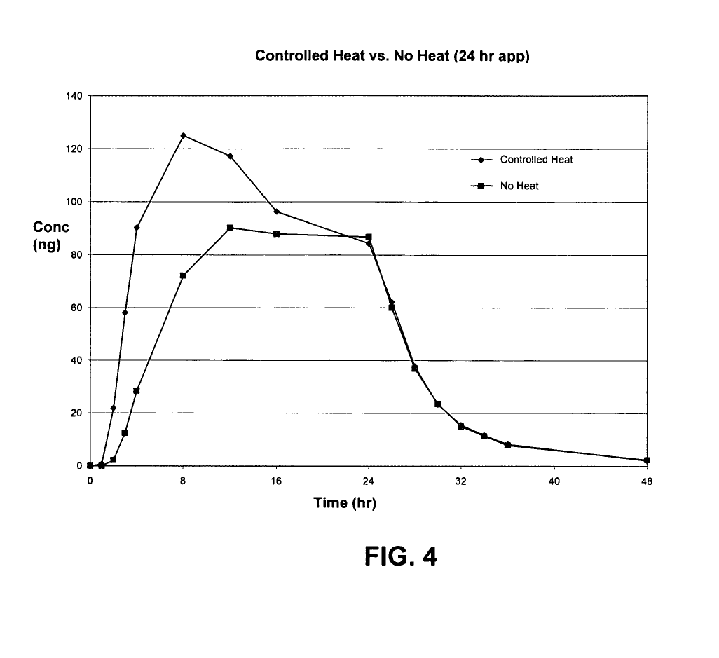

Ketoprofen patches, each with 100 cm² surface area, were administered to the back area of two groups of human subjects. One group (13 subjects) received the patch without heating. The other group (12 subjects) received the patch with an exothermic heating apparatus which kept the mean skin temperature in the range of 38 to 42° C. for more than 6 hours. Concentrations of ketoprofen in blood samples taken at specific time intervals were measured and are shown in FIG. 4 (Mean of the all subjects in each group). Although the target will typically be the tissues of the knee or other joints, drug concentrations in blood circulation based on application to the back in this testing protocol are believed to be a good measure of how much drug is delivered across the skin in general. As can be seen in FIG. 4, controlled heating significantly increased the transdermal delivery of ketoprofen, especially in the early hours.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A system for dermal delivery of a drug, comprising,
a heating apparatus including at least two heating elements, said heating elements each including an exothermic chemical composition, wherein the heating element is exposed to ambient oxygen through a cover, said cover controlling the amount of ambient oxygen capable of contacting the chemical composition; and
a drug-containing layer including a drug and having a delivery surface with an area of about 50 cm² to about 400 cm² and a drug.

2. A system as in claim 1, wherein the drug-containing layer includes a back surface opposite the delivery surface, and wherein said heating elements collectively cover from about 20% to about 80% of a surface area of the back surface.

3. A system as in claim 1, wherein the cover of the heating apparatus is a sheet of material having a pre-determined air permeability that controls the oxygen flow from ambient air to each heating element.

4. A system as in claim 1, wherein the cover of the heating apparatus comprises an oxygen impermeable material having holes therethrough which allow the oxygen to contact each heating element.

5. A system as in claim 4, wherein the total area of all the holes in the cover is about 0.5% to about 2.5% of the total surface area of the cover that is accessible to the exothermic chemical composition.

6. A system as in claim 4, wherein the total area of all the holes in the cover is about 1.0% to about 2.0% of the total surface area of the cover that is accessible to the exothermic chemical composition.

7. A system as in claim 4, wherein the total area of all the holes in the cover is about 1.2% to about 1.8% of the total surface area of the cover that is accessible to the exothermic chemical composition.

8. A system as in claim 4, wherein each heating element has exposure to ambient oxygen only through holes, each with diameter of at least 0.05 inch.

9. A system as in claim 4, wherein each heating element is exposed to the ambient oxygen through from 2 to 10 holes present in the cover.

10. A system as in claim 1, wherein the heating elements are enclosed in separate chambers that are separated within the device from one another such that oxygen flow into one chamber cannot flow to other adjacent chambers.

11. A system as in claim 1, wherein all of the heating elements are enclosed in a single chamber such that oxygen flow into the chamber can reach other heating elements within the chamber.

12. A system as in claim 1, wherein some of the heating elements are enclosed in a single chamber such that oxygen flow into the chamber can reach other heating elements within the chamber, and at least one of the heating elements is enclosed in a separate chamber that is separated within the device from other heating elements such that oxygen flow to another adjacent chamber.

13. A system as in claim 1, wherein each heating element is substantially an elliptical shape.

14. A system as in claim 1, wherein the heating apparatus includes from 2 to 20 heating elements.

15. A system as in claim 1, wherein the heating apparatus includes from 3 to 8 heating elements.

16. A system as in claim 1, wherein the heating apparatus includes from 4 to 6 heating elements.

17. A system as in claim 1, wherein the heating elements are arranged in a singular row.

18. A system as in claim 1, wherein the heating elements are arranged in two rows.

19. A system as in claim 1, wherein the heating elements are arranged in three or more rows.

20. A system as in claim 1, wherein the heating elements are arranged in pattern that is non-linear and that is ergonomically configured for application over a specific joint.

21. A system as in claim 1, wherein the heating elements are substantially flattened, and one side of each heating element has a surface area of 5 cm² to 25 cm².

22. A system as in claim 1, wherein the heating elements are substantially flattened, and one side of each heating element has a surface area of 8 cm² to 18 cm².

23. A system as in claim 1, wherein the heating elements are substantially flattened, and one side of each heating element has a surface area of 11 cm² to 15 cm².

24. A system as in claim 1, wherein the drug is an anti-inflammatory agent.

25. A system as in claim 1, wherein the drug is an NSAID selected from the group consisting of ketoprofen, diclofenac, salicylates, arylalkanoic acids, profens, fenamic acids, pyrazolidine derivatives, oxicams, COX-2 inhibitors, sulphonanilides, licofelone, omega-3 fatty acids, and combinations thereof.

26. A system as in claim 1, wherein the drug includes ketoprofen.

27. A system as in claim 1, wherein the drug includes diclofenac.

28. A system as in claim 1, wherein the drug includes lidocaine.

29. A system as in claim 1, wherein the drug-containing layer has a drug delivery surface having an area of 75 cm² to 250 cm².

30. A system as in claim 1, wherein the drug-containing layer has a drug delivery surface having an area of 150 cm² to 200 cm².

31. A system as in claim 1, wherein the heating apparatus and the drug-containing layer are separately stored and are combined immediately prior to use.

32. A system as in claim 1, wherein the exothermic chemical composition comprises iron, activated carbon, salt, and water.

33. A system as in claim 32, wherein the exothermic chemical composition further comprises a sulfur-containing compound.

34. A system as in claim 33, wherein the sulfur-containing compound is selected from the group consisting of elemental sulfur, sulfates, sulfites, sulfides, thiosulfates, and combinations thereof.

35. A system as in claim 32, wherein the weight ratio of water to all other ingredients in the exothermic chemical composition is about 1:2 to about 1:8

36. A system as in claim 32, wherein the weight ratio of water to all other ingredients in the exothermic chemical composition is about 1.0:2.3 to about 1.0:4.5.

37. A system as in claim 1, wherein each heating element is capable of providing heat for a duration of at least 2 hours.

38. A system as in claim 1, wherein each heating element is capable of providing heat for a duration of at least 6 hours.

39. A system as in claim 1, wherein each heating element is capable of providing heat for a duration of at least 10 hours.

40. A system as in claim 1, wherein the system is configured to remain adhered to a body surface for a period greater than about 6 hours

41. A system as in claim 40, wherein the body surface is a knee.

42. A system as in claim 40, wherein the body surface is a back or neck.

43. A system as in claim 40, wherein the body surface is an elbow, shoulder, wrist, or ankle.

44. A system as in claim 40, wherein the heating apparatus and drug-containing layer are configured to remain adhered to a skin surface for a period greater than about 10 hours even when said body surface is subjected to active stretching and movement.

45. A system as in claim 1, wherein the drug-containing layer has rounded corners with a radius of 2 to 10 cm.

46. A system as in claim 1, wherein the length to width ratio of the drug-containing layer is about 1.2:1 to about 5:1.

47. A system as in claim 1, wherein the heating apparatus and the drug-containing layer are part of an integrated device.

48. A system as in claim 47, wherein the heating apparatus and the drug-containing layer are physically separated by a barrier layer located between the heating apparatus and the drug-containing layer, said barrier configured to prevent migration of the drug from the drug-containing layer into the heating apparatus.

49. A system as in claim 47, wherein the barrier layer includes a metallic layer.

50. A system as in claim 49, wherein the metallic layer provides lateral homogenization of heat from the heating elements to the drug-containing layer.

51. A system as in claim 49 wherein the metallic layer includes a metal foil.

52. A system as in claim 49 wherein the metal foil has a thickness of from 2.5 micrometers to 260 micrometers.

53. A system as in claim 49 wherein metallic layer is laminated with a polymeric layer on both sides thereof.

54. A system as in claim 49, wherein the barrier layer comprises three layers of material, two independently selected outer layers and a center layer.

55. A system as in claim 54, wherein the center layer is a metal foil.

56. A system as in claim 55, wherein at least one of the outer layers is configured to prevent rusting of the metal foil.

57. A system as in claim 54, wherein at least one of the layers is a polymeric film.

58. A system as in claim 54, wherein at least one of the layers is an adhesive.

59. A system as in claim 49, wherein the metallic layer includes a material selected from aluminum, steel, copper, tin, nickel, and alloys and mixtures thereof.

60. A system as in claim 49, wherein the metallic layer includes aluminum.

61. A system as in claim 49, wherein the metallic layer consists essentially of aluminum.

62. A system as in claim 49, wherein each of the heating elements is separated from the metallic layer by a barrier material substantially impermeable to water.

63. A system as in claim 49, wherein the metallic layer is configured to break during normal use to provide more flexibility when applied to stretchable skin.

64. A system as in claim 49, wherein the metallic layer is laminated on both sides with a polymeric layer.

65. A system as in claim 64, wherein the polymeric layers are configured to provide adhesion between the metallic layer and the drug-containing layer and the heating apparatus.

66. A system for dermal delivery of a drug, comprising,
a drug-containing layer comprising an NSAID, said drug-containing layer including a drug delivery surface having an area of about 50 cm² to about 400 cm² and formulated to provide dermal delivery of the NSAID; and
a heating apparatus including at least one heating element, said heating element including an exothermic chemical composition, wherein said heating element is configured to be exposed to ambient oxygen through a cover, said cover controlling the amount of ambient oxygen capable of contacting the chemical composition, wherein the heating apparatus is configured to provide controlled heating;
wherein the heating apparatus is configured to be connected to or disposed proximate to the drug-containing layer such that when placed on a human body surface of a subject experiencing musculoskeletal pain or inflammation, the controlled heating from the heating apparatus and the dermal delivery of the NSAID from the drug-containing layer provides greater relief to the subject defined by less inflammation or less pain retained by the subject compared to when either dermal delivery of the NSAID or application of the controlled heating is administered alone.

67. A system as in claim 66, wherein the controlled heating from the heating apparatus combined with the NSAID provide a synergistic effect.

68. A system as in claim 66, wherein at least one portion of the NSAID is delivered into the target tissues of the subject suffering from musculoskeletal pain or inflammation through regional delivery.

69. A system as in claim 66, wherein the heating apparatus and the drug-containing layer are integrated.

70. A system for treating musculoskeletal pain or inflammation, comprising:
an elastic air impermeable cover including a plurality of holes therethough;
at least two heating elements positioned beneath the cover;
a polymeric layer positioned beneath the heating elements, and sealed to the cover to provide one or more chamber for the heating elements;
a metal barrier positioned beneath the polymeric layer; and
a drug-containing layer including a drug delivery surface having an area of about 100 cm² to about 250 cm²,
wherein the metal barrier is configured to prevent migration of the drug into the heating apparatus.

71. A system as in claim 70, further comprising a second polymeric layer interposed between the metal barrier and the drug-containing layer.

72. A system for treating musculoskeletal pain or inflammation, comprising:
a heating apparatus comprising at least five heating elements each with a surface area on one side from about 12 cm² to 14 cm², said heating elements each including an exothermic chemical composition, wherein each heating element is exposed to ambient oxygen through a cover, said cover comprising a material that is air-impermeable and having at least six holes associated specifically with each of the at least five heating elements, wherein the holes have a diameter from about 0.065 inch to about 0.085 inch; and
a drug-containing layer comprising ketoprofen and having a surface area of about 150 cm² to about 200 cm²;
wherein the heating apparatus is configured to maintain the skin at a temperature from about 38° C. to about 42° C. for at least 6 hours.

73. A system as in claim 72, wherein the heating apparatus has exactly five heating elements.

74. A system as in claim 72, wherein the heating elements have exactly six holes associated specifically with each of the at least five heating elements.

75. A method of treating musculoskeletal pain or inflammation, comprising applying a system to a body surface under which musculoskeletal pain or inflammation exists, said system comprising a heating apparatus including a heating element, said heating element including an exothermic chemical composition, wherein each heating element is exposed to ambient oxygen through a cover, said cover controlling the amount of ambient oxygen capable of contacting the chemical composition; and a drug-containing layer including a drug and having a delivery surface with an area of about 50 cm² to about 400 cm² and a drug.

76. A method as in claim 75, wherein the body surface is an active body surface.

77. A method as in claim 76, wherein the active body surface is a knee.

78. A method as in claim 75, wherein the heating apparatus includes at least two separate heating elements.

79. A method as in claim 75, wherein the drug includes diclofenac.

80. A method as in claim 75, wherein the drug includes lidocaine.

81. A method as in claim 75, wherein the drug includes ketoprofen.
